(19) United States
(12) Patent Application Publication
(10) Pub. No.: US 2009/0280195 A1
(43) Pub. Date: Nov. 12, 2009

(54) Methods for the treatment of addictive disease and addiction to chemical substances and addictive behaviors using a formula containing the following formulation: Mucuna pruiens, N Acetyl L Cysteine, Magnesium Citrate

(76) Inventor: Thomas Eugene Hoshour, Indianapolis, IN (US)

Correspondence Address: Thomas Eugene Hoshour, 7349 NORTH PENNSYLVANIA STREET, INDIANAPOLIS, IN, 46240, US

(21) Appl. No.: 12/151,395

(22) Filed: May 7, 2008

(51) Int. Cl. A61K 36/00; A61P 25/30

(52) U.S. Cl. 424/725

(57) ABSTRACT

The invention is directed at the formulation of Mucuna pruiens, N Acetyl L Cysteine and Magnesium Citrate. This formulation augments dopamine in the striatal area of the human brain and stabilizes membranes by restoring normal neurotransmitter levels and decreasing endogenous catecholamines. These effects are beneficial in the short and long term treatment of addictive disease and addiction to chemical substances and addictive behaviors.

### FIELD OF THE INVENTION

The herbal compound, supplement and mineral formula claims multiple benefits in the treatment of addictive disease. Addictive disease in this context will relate to an addiction to chemical substances or addictive behaviors.

The herbal compound, supplement and mineral formula abates signs and symptoms of acute and chronic withdrawal and the cravings that are associated with the addiction to chemical substances and addictive behaviors. This is accomplished by augmenting certain neurotransmitters in the central nervous system and stabilizing membranes in the central nervous system.

### BACKGROUND OF THE INVENTION

It is well known that addiction to chemical substances and addictive behaviors are mediated through the pleasure center and medial forebrain bundle of the brain. Addictive substances and addictive behaviors cause an increase in dopamine in the area of the nucleus accumbens and prefrontal cortex resulting in a euphoric response. The increase in dopamine during the addictive process also exerts its effects through the mesolimbic and mesocortical projections of the medial for brain bundle. With ongoing addictions there are changes in:

Neurotransmitter levels

Cell surface events

Secondary messenger systems

Receptor site sensitivity

Electrical and chemical balance of the brain

### Withdrawal and Cravings from Addictive Disease

Withdrawal and cravings from addictive substances and addictive behaviors are a result of absences of the addictive substance or addictive behavior. A major portion of the withdrawal signs and symptoms is a consequence of decreased dopamine levels in the mesolimbic and mesocortical systems of the medial fore-brain bundle. There are also alterations in the levels of the neurotransmitters. Nor-epinephrine is commonly released in withdrawal. This commonly increases blood pressure and heart rate. This also results in hyper vigilance causing anxiety, restlessness, and a general dysphoria. Ongoing withdrawal and cravings are also a result of disruption or receptor sites where the receptor site is either up or down regulated. This, along with the dysregulation of the neurotransmitter levels describes the phenomena of the “kindled brain model” seen in ongoing addictive disease. The “kindled brain” is essentially the result of damage to the electrical and chemical systems of the brain. This also destabilizes membranes in the brain. The combined effect result in restlessness, agitation and mood swings that commonly result in relapse back into addictive disease. Thus, withdrawal and cravings are commonly the result of:

1. Decreased stimulation or the pleasure center of the brain. Specifically the mesocortical and mesolimbic radiations and those areas responsible for pleasure and euphoria in the nucleus accumbens and pre-frontal cortex.

2. Decreased dopamine levels in those areas mentioned above.

3. Alterations in the neurotransmitters levels.

4. Increased levels of nor-epinephrine resulting in restlessness, hypervigilance, and possible increased heart rate, blood pressure and central nervous system stimulation.

5. Destabilization of membranes secondary to down and up regulation of receptor site sensitivities, and disruptions of the chemical and electrical balance of the brain.

6. The “kindled brain model” which describes a brain in which the electrical and chemical balance has been disrupted. This results in episodic cravings, restlessness, and mood swings.

Current treatment for addictive disease is lacking in the medical community. Insurance companies frequently provide little or no coverage for the mental health issues or addictive disease. There is a paucity of trained personnel to help individuals with addictive disease. Frequently, affected individuals have no where to turn and traditional medical care for addictive disease is expensive and intimidating to the patient

Individuals deserve the right to have a choice in their treatment. This herbal/supplement/mineral formula provides individuals that choice. This formula provides the suffering addict a cost effective means of treatment and a means to maintain their anonymity.

### SUMMARY OF THE INVENTION

A method to treat addictive disease based on current medical principals are clearly indicated. This method should have minimal side effects, be readily accessible and cost effective.

A method to treat addictive disease using a formula of Mucuna pruiens, N Acetyl L Cysteine, and Magnesium Citrate provides this method. The composition of this invention is designed to have particular efficacy for the treatment of addictive disease as it relates to addiction to stimulants, nicotine, hallucinogens, eating, gambling, drug dealing and compulsive spending.

The invention provides a unique approach to the treatment of addictive disease by augmenting dopamine and other neurotransmitters Involved with mood in the area of the prefrontal cortex nucleus accumbens and medial fore-brain bundle. The invention also stabilizes membranes through GABA-ergic systems and other inhibitory mechanisms. The net result of these actions is an elevation of mood, sense of well-being and attenuation of the common signs and symptoms of withdrawal.

The invention provides a method of treatment for addictive disease in the following situations:

1. Acute withdrawal from addicting chemical substances or behaviors.

2. Chronic withdrawal from addicting chemical substances or behaviors.

3. Acute cravings for addicting chemical substances and behaviors.

4. Chronic cravings for addicting chemical substances or behaviors.

5. The “kindled brain” with the chemical and electrical changes due to ongoing addictive disease.

An objective of the invention is to provide a convenient way for individuals to consume the formulation comprising Mucuna pruiens, N Acetyl N Cysteine, and Magnesium Citrate.

An objective of the invention is to provide an optimal composition of the formulation of the invention where in each component of the formula is present in an optimal quantity represented in milligrams. Mucuna pruiens 100 milligrams, N Acetyl L Cysteine 500 milligrams, and Magnesium Citrate 100 milligrams. The dose of each medicant may be higher or lower than the dose which is presented in this document

Another objective of the invention is to provide a convenient method for the individuals to consume the formulation comprising Mucuna pruiens 100 milligrams, N Acetyl L Cysteine 500 milligrams, and Magnesium Citrate 100 milligrams. This was accomplished by providing a tablet which contained these three medicants In the quantities: Mucuna pruiens 100 milligrams, N Acetyl L Cysteine 500 milligrams and Magnesium Citrate 100 milligrams. The psychoactive portion of Mucuna pruiens is derived from the bean of its plant. The N Acetyl L Cysteine and Magnesium Citrate are provided as powders. The filler comprises 15% of the tablet weight. This tablet is to be taken with food or water.

Yet another objective of this invention is to provide a treatment protocol where the individuals would be able to follow dosing recommendations to treat addictive disease based on the severity of their addictive disease. This was accomplished by providing a treatment protocol for the individuals who use the invention to treat their addictive disease.

### DESCRIPTION OF THE PREFERRED EMBODIMENTS

This formula containing Mucuna pruiens 100 milligrams, N Acetyl L Cysteine 500 milligrams and Magnesium Citrate 100 milligrams is designed to treat acute and chronic withdrawal, acute and chronic cravings, and distorted chemical and electrical balances associated with addictive disease. This formula is designed treat addictions to stimulants, nicotine, hallucinogens, eating, gambling, drug dealing, and compulsive spending. The compounds and composition of the formula to treat addictive disease to stimulants, nicotine, hallucinogens, eating, gambling drug dealing, and compulsive spending was selected for the following reasons:

### Mucuna pruiens

1. Mucuna pruiens contains a significant amount of L Dopamine. L Dopamine crosses the blood brain barrier. L Dopamine becomes dopamine in the brain and this increases dopamine augmentation in the strietel area including the prefrontal cortex nucleus accumbens, meso-limbic and meso-cortical areas. This is helpful in treating addictive disease. These sections of the brain are commonly deficient in dopamine during detoxification, withdrawal and cravings from stimulants, nicotine, hallucinogens, eat, gambling, drug dealing, and compulsive spending.

2. Mucuna pruiens increases dopamine levels. This is helpful in treating the depressed mood and ahedonia common with detoxification, withdrawal, and cravings from stimulants, nicotine, hallucinogens, eating, gambling, drug dealing, and compulsive spending.

3. Mucuna pruiens has antioxidant properties. This helps to reduce the free radicals created during detoxification, withdrawal, and cravings.

4. Mucuna pruiens increases L Dopamine which increases dopamine which in turn, increases growth hormone releasing factor from the hypothalamus which increases growth hormone. Growth hormone helps in detoxification, withdrawal, and cravings by helping the body maintain an anabolic state.

5. Mucuna pruiens helps to normalize movement, sexuality and mood. This is helpful in treating detoxification, withdrawal and cravings from stimulants, nicotine, hallucinogens, eating, gambling, drug dealing and compulsive spending.

6. Mucuna pruiens increases hormone sensitive lipase. This assists in burning fatty acids for fuel. This can be helpful in detoxification, withdrawal and cravings from stimulants, nicotine, hallucinogens, eating, gambling, drug dealing and compulsive spending.

7. Mucuna pruiens helps to prevent adrenal fatigue. This is helpful In treating detoxification, withdrawal, and cravings from stimulants, nicotine, hallucinogens, eating, gambling, drug dealing and compulsive spending. These addictions are stressful to the adrenal gland.

8. Mucuna pruiens has a neuro-protective effect. This is helpful in detoxification, withdrawal and cravings as it decreases “firing” in the nervous system and stabilizes membranes.

9. Mucuna pruiens has an antidepressant effect. This is helpful in normalizing the mood which is commonly depressed in detoxification, and withdrawal from stimulants, nicotine, hallucinogens, eating, gambling, drug dealing, and compulsive spending.

10. Mucuna pruiens has a neuro-restorative effect and helps to restore normal levels of L Dopamine, dopamine, nor-epinephrine and serotonin. This is helpful in detoxification, withdrawal and cravings associated with stimulants, nicotine, hallucinogens, eating, gambling, drug dealing and compulsive spending. Addiction commonly alters levels of theses neurotransmitters.

11. Mucuna pruiens inhibits the prolactin response. This increases L Dopamine and helps with normal sexual function. This is helpful in detoxification, withdrawal and cravings.

12. Mucuna pruiens helps to stabilize carbohydrate metabolism. This is helpful in detoxification, withdrawal, and cravings by normalizing appetite and decreases “comfort eating.”

13. Mucuna pruiens increases growth hormone and androgenic activity. This is helpful in detoxification, withdrawal and cravings to maintain a lean body mass.

14. Mucuna pruiens increases libido. This is helpful in detoxification withdrawal and cravings due to the fact that libido is generally depressed during these periods.

15. Mucuna pruiens at low doses is a stimulant which decreases sleeping time and increases motor activity. This is helpful in the treatment of detoxification, withdrawal and cravings from stimulants, nicotine, hallucinogens, eating, gambling, drug dealing and compulsive spending.

16. Many of the actions of N Acetyl L Cysteine are synergistic with Mucuna pruiens in treating detoxification, withdrawal and cravings associated with addiction to stimulants, nicotine, hallucinogens, eating, gambling, drug dealing, and compulsive spending.

17. Many of the actions of Magnesium Citrate are synergistic with Mucuna pruiens in treating the detoxification, withdrawal and cravings associated with addiction to stimulants, nicotine, hallucinogens, eating gambling, drug dealing and compulsive spending.

### N Acetyl L Cysteine

1. N Acetyl L Cysteine increases the activity of a protein which functions as a Cysteine glutamate exchanger and restores glutamate levels which are decreased during addiction. This helps in detoxification, withdrawal and cravings. This occurs in the area of the nucleus accumbens which is a major portion of the brain involved in addiction. normalizing extra cellular glutamate levels decreases neuro-excitability and decreases the signs and symptoms of detoxification, withdrawal and cravings.

2. N Acetyl L Cysteine is a precursor of glutathione, a critical antioxidant. Antioxidants neutralize free radicals which are formed during detoxification, withdrawal and cravings associated with addictive disease.

3. N Acetyl L Cysteine stabilizes glutamate transmission and restores extra cellular concentrations of glutamate which is altered during addiction. Glutamate is associated with the reward system. restoring normal extra cellular concentration of glutamate is beneficial in treating detoxification, withdrawal, and cravings associated with addictive disease by normalizing the disrupted pleasure center of the brain.

4. N Acetyl L Cysteine stabilizes glutamate transmission and restores extra cellular concentration of glutamate which was altered during addiction. This decreases neuro-excitability. Neuro-excitability is a major problem seen in detoxification, withdrawal, and cravings In addictive disease. N Acetyl L Cysteine is helpful in treating these problems.

5. N Acetyl L Cysteine is hepato-protective. This is helpful in treating detoxification, withdrawal, and cravings associated with addictive disease. The liver is commonly adversely affected by addictive disease.

6. Many of the actions of N Acetyl L Cysteine and Mucuna pruiens are synergistic in treating detoxification, withdrawal and cravings associated with addictive disease.

7. Many of the actions of Magnesium Citrate are synergistic with N Acetyl L Cysteine in treating the detoxification, withdrawal and cravings associated with addictive disease.

### Magnesium Citrate

1. Magnesium decreases neuromuscular excitability that is commonly seen in the detoxification, withdrawal, and cravings associated with addictive disease.

2. Magnesium decreases the risk of seizure and neuro-excitability that are seen in the detoxification, withdrawal, and cravings seen in addictive disease.

3. Magnesium decreases endogenous catecholamine release. Excess catecholamines are common problems in the detoxification, withdrawal and cravings in individuals with addictive disease.

4. Magnesium has an antagonistic affect on N Methyl D Aspartate receptor sites. N Methyl Aspartate receptor sites are excitatory in nature. Down regulating N Methyl D Aspartate receptor sites would be helpful in treating the detoxification, withdrawal, and cravings commonly seen in addictive disease.

5. Magnesium has neuro-protective effect. This would be beneficial during detoxification, withdrawal and cravings that is commonly seen in addictive disease.

6. Many of the actions of Mucuna pruiens are synergistic with Magnesium Citrate in treating the detoxification, withdrawal and cravings associated with addictive disease.

7. Many of the actions of N Acetyl L Cysteine are synergistic with Magnesium Citrate in treating the detoxification, withdrawal, and cravings associated with addictive disease.

This invention provides a formula containing an herb, an amino acid, and a mineral. The composition of this formula has been created to treat addiction to chemical substances: stimulants, nicotine, hallucinogens, and addictive behaviors of eating, gambling, drug dealing and compulsive spending. This invention has been founded on the scientific principals I have set forth in this paper. The formula and the composition of the formula contains 100 milligrams Mucuna pruiens. The composition of the formula also contains the amino acid N Acetyl L Cysteine 500 milligrams. Also included in the formulation is Magnesium Citrate 100 milligrams. This formula is unique and different from all other herbal supplement mineral formulas for the following reasons:

1. The composition of the formula Mucuna pruiens, N Acetyl L Cysteine and Magnesium Citrate were chosen specifically to treat addictions to chemical substances and addictive behavior for the reasons I have already discussed. The mechanisms of action and the scientific reasoning for the composition of this invention provides sound scientific fact for the treatment of addictive disease. This is in stark contrast to other formulas and supplements for treatment of addiction which make sweeping claims but no rationale or scientific basis for these claims. There is currently no formula with the composition of this invention being promoted for treatment of addictive disease.

2. The composition of the formula uses low doses of Mucuna pruiens to achieve a stimulatory effect that is helpful in treating addiction withdrawal and cravings for stimulants, nicotine, hallucinogens, eating gambling, drug dealing and compulsive spending.

3. The composition of the formula is designed to treat the signs and symptoms of acute and chronic withdrawal, and acute and chronic cravings. The treatment protocol put forth with this formula appreciates the intensity and duration of these problems and doses appropriately.

4. The composition of the formula provides synergistic actions in the treatment of addictive disease. These mechanisms of action have been described. Other formulas have no comparable mechanisms or similar rationale for the treatment of addictive disease.

5. The composition of this formula targets specific parts of the human brain which are thought to be responsible for addictions.

6. The composition of this formula augments specific neuro-transmitters which have been altered during the course of addiction and stabilizes the membranes within the central nervous system that have been destabilized through the addictive process.

7. The treatment protocol provided with this invention addresses the need to augment certain neurotransmitters early on in withdrawal and to stabilize membranes. The treatment protocol also address the need for long term treatment and the necessity to slowly decrease the medicants in order for the central nervous system to regain homeostasis. No other formulas for addictive treatment utilize this methodology.

8. The composition of the formula and this treatment protocol addresses the “kindled brain model” of addictive disease. The “kindled brain” describes a situation where addiction has created long term chemical and electrical alterations of the brain.

9. The treatment protocol provided with this invention addresses the treatment relative to level of addictive disease; mild, moderate or severe. Other formulas do not provide treatment protocols for varying levels of addictive disease.

10. The composition of the formula targets those areas of the brain responsible for the addictive process to stimulants, nicotine, hallucinogens, eating, gambling, drug dealing and compulsive spending.

11. The composition of the formula targets restoration of specific neurotransmitters and hormone levels which were altered during the course of addiction to stimulant, nicotine, hallucinogens, eating, gambling, drug dealing and compulsive spending.

12. This formula has been selected due to the medicants' mechanism of action which have been addressed in this document. These mechanisms of action have been proven to ameliorate the signs and symptoms of addictive disease relative to the detoxification, withdrawal, and cravings. This provides legitimate indications for the use of this formula. This is a stark contrast to other herbal-supplement formulas that make broad sweeping statements with no scientific basis.

13. This formula provides a combination of medicants which are specifically effective in treating addictive disease to chemical substances and addictive behaviors. This formula and the particular combination has not been used before in this manner because the concepts set forth here were not understood.

14. The logic and rationale for the combination and dosing for this formula was unknown until this invention elucidated the reasoning for the combinations and dosing.

15. The combinations and dosing of this formula are useful for the treatment of addiction to the chemicals substances and addictive behaviors previously described in this document.

16. The dosing protocol set forth in this invention is unique and different in many respects:
the protocol addresses the need to augment specific neurotransmitters early on in the detoxification and withdrawal from addicting chemical substances and addicting behaviors.
the protocol addresses the need to titrate the dose of the formula over time to avoid untoward negative feedback responses with endogenous neurotransmitters and other neurochemicals.
the protocol addresses the need to have “as needed” (prn) dosing to address the episodic problems that are so common in addictive disease. This is a totally unique feature or the invention.
the protocol addresses the fact that the treatment of addictive disease can require at least eighteen months or longer. The majority of treatments for addictive disease range from seventy two hours to five days.
the protocol addresses the need to treat the “kindled brain model” of the addictive disease. This model describes a brain which has developed chemical and electrical imbalances as a result of ongoing addictive disease.

The formula has been selected for specific uses and for specific reasons which have been elucidated in this document. This is in contrast to other herbal supplement formulas that make broad sweeping statements with no scientific basis. The formula and protocol provide a means of treatment for acute and chronic detoxification and withdrawal as well as acute and chronic cravings associated with addictive disease.

This formula and protocol provide a means to treat addictive disease in all its phases; acute/chronic detoxification and withdrawal; acute/chronic cravings, and the kindled brain model. This formula and protocol also provides a means to treat addictive disease by level of intensity: mild, moderate and severe.

### Treatment Protocol

### For

### Detoxification, Withdrawal, and Cravings

### From

Stimulants, nicotine, hallucinogens, eating, gambling, drug dealing and compulsive spending using a formula containing

### Recovery for Life Formula One

### Mucuna pruiens 100 milligrams

### N Acetyl L Cysteine 500 milligrams

### Magnesium Citrate 100 milligrams

And a formula containing

### Recovery for Life Craving Aid

### Glutamine 250 milligrams

### B6 (pyridoxine) 10 milligrams

### Alternative Medicine Herbal Treatment Outline

Formula I: treatment for alcohol-drugs-addictive behaviors.

*Detoxification *Withdrawal *Cravings

### Detoxification/Withdrawal Severe Moderate Mild

### Detoxification/Withdrawal Severe

Recovery for Life Formula I
1-2 tablets 4 times a day for 14 days

Recovery for Life Craving Aid
1-2 tablets every 3 hours for cravings not to exceed 8 tablets in 24 hours for 14 days

Recovery for Life Formula I
1 tablet 3 times daily for 14 days

Recovery for Life Craving Aid
1-2 tablets every 4 hours for cravings, not to exceed 8 tablets in 24 hours for 14 days

Recovery for Life Formula I
1 tablet 2 times daily for 18 months minimum and as needed

Recovery for Life Craving Aid
1-2 tablets every 6 hours for cravings not to exceed 8 tablets in 24 hours for a period of 18 months minimum and as needed

### Detoxification/Withdrawal Moderate

Recovery for Life Formula I
1 tablet 3 times daily for 10 days

Recovery for Life Craving Aid
1-2 tablets every 4 hours for cravings not to exceed 8 tablets in 24 hours for 10 days

Recovery for Life Formula I
1 tablet 2 times daily for 18 months minimum and as needed

Recovery for Life Craving Aid
1-2 tablets every 6 hours for cravings not to exceed 8 tablets in 24 hours for 18 months minimum and as needed

### Detoxification/Withdrawal Mild

Recovery for Life Formula I
1 tablet 2 times daily for 18 months minimum and as needed

Recovery for Life Craving Aid
1-2 tablets every 6 hours for cravings not to exceed 8 tablets in 24 hours for 18 months minimum and as needed

Cravings Acute

Recovery for Life Formula I
Increase dosage by one tablet daily to a maximum of 1 tablet 4 times daily. Do this at one week intervals. After cravings have subsided, may decrease dosage by one tablet daily at one week intervals. Would stay at 1 tablet 2 times daily for at least 18 months and as needed thereafter May repeat this treatment as needed

Recovery for Life Craving Aid
1-2 tablets every 3 hours for cravings not to exceed 8 tablets in 24 hours. After cravings have subsided may decrease dosage as needed. May repeat this treatment as needed.

### Cravings Chronic

Recovery for Life Formula I
Increase dosage by one tablet daily to a maximum of 1 tablet 4 times a day. Do this at 1 week intervals. Would stay at 1 tablet 2 times daily for at least 18 months and as needed. May repeat this treatment as needed

Recovery for Life Craving Aid
1-2 tablets every 6 hours for cravings not to exceed 8 tablets in 24 hours. After cravings have subsided may decrease dosage as needed. May repeat this treatment as needed

1. A formula comprising an herb, supplement and mineral that is effective In treating addictive disease in humans.

2. The formula composition according to claim 1, where the treatment addresses acute and chronic detoxification and withdrawal; acute and chronic cravings, as well as electrical and chemical alterations of the brain due to addictive disease.

3. A formula composition according to claim 1, which contains an herb, amino acid and a mineral.

4. A formula composition according to claim 3, where in said herb comprises Mucuna pruiens.

5. A formula composition according to claim 3, where in said amino acid is composed of N Acetyl L Cysteine.

6. A formula composition according to claim 3, where in said mineral comprises Magnesium Citrate.

7. A formula composition according to claim 4, where in Mucuna pruiens is present at 100 milligrams. Dose may vary.

8. A formula composition according to claim 5, where in magnesium citrate is present at 100 milligrams. Dose may vary.

9. A formula composition according to claim 6, where in Magnesium Citrate is present at 100 milligrams. Dose may vary.

10. A formula for the treatment of addictive disease comprising:
Mucuna pruiens 100 milligrams; dose may vary.
N Acetyl L Cysteine 500 milligrams; dose may vary.
Magnesium citrate 100 milligrams; dose may vary.

11. A formula for the treatment of addictive disease according to claim 10, is provided in a tablet for oral ingestion with water or food.

12. A formula for the treatment of addictive disease according to claim 11, which provides a treatment for addictive disease involving stimulants, nicotine, hallucinogens, eating, gambling, drug dealing and compulsive spending.

13. A formula for the treatment of addictive disease according to claim 12, where by the frequency and duration of the tablets ingested is dependent on the severity of the addictive disease involving stimulants, nicotine, hallucinogens, eating, gambling, drug dealing and compulsive spending.
